(19) United States
(12) Patent Application Publication
(10) Pub. No.: US 2005/0070804 A1
(43) Pub. Date: Mar. 31, 2005

(54) Cuff and blood pressure measuring apparatus

(76) Inventor: Daisy Han, Taipei (TW)

Correspondence Address: J C PATENTS, INC., 4 VENTURE, SUITE 250, IRVINE, CA, 92618, US

(21) Appl. No.: 10/777,264

(22) Filed: Feb. 11, 2004

(30) Foreign Application Priority Data
Sep. 26, 2003 (TW) 92217312

(51) Int. Cl. A61B 5/02

(52) U.S. Cl. 600/490; 600/499

(57) ABSTRACT

A cuff and a blood pressure measuring apparatus using thereof are disclosed. The blood pressure-measuring apparatus essentially comprises a blood pressure display unit, a cuff and a pressure sensing unit. The cuff essentially comprises a strap unit and a ring. The ring is embedded in the strap unit, and the direction of the ring is not perpendicular to the outstretch direction of the strap unit. The strap unit is adapted to pass through the ring. The blood pressure sensing unit is disposed on the strap unit, for sensing the pressure inside the cuff.

### CROSS-REFERENCE TO RELATED APPLICATION

This application claims the priority benefit of Taiwan application serial no. 92217312, filed on Sep. 26, 2003.

### BACKGROUND OF THE INVENTION

1. Field of the Invention

The present invention relates to a cuff and a blood pressure measuring apparatus using the cuff. More particularly, the present invention relates to a cuff that is ergonomically designed and can be firmly fitted on the body extremity of a subject to increase the blood pressure measuring accuracy.

2. Description of Related Art

In recent years, the leading causes of death in the nation includes, malignant neoplasm, cerebral vascular disease, cardiovascular disease, unintentional injury, diabetes mellitus, chronic liver disease and heptaocirrhosis, nephritis and nephritis, pneumonia, bronchitis, hypertension-related diseases, emphysema, asthma, tuberculosis, etc. Among these various diseases, a majority of them, for example, cerebral vascular disease, cardiovascular disease, hypertension-related disease, diabetes mellitus and nephropathy, which are the second, fourth, fifth, seventh and ninth leading causes of death, is directly or closely related to hypertension. About 18% of the population at the age of 40 and above in this nation suffers from hypertension. It is apparent that hypertension poses a great threat to health. Hence, proper and adequate prevention of hypertension in the elderly population is very crucial.

According to one of the standards of the World Health Organization (WHO), hypertension is defined by a systolic pressure above 160 mmHg and a diastolic pressure above than 95 mmHg. Currently, about seven hundred millions people, i.e. about 12% of the world's population of the six thousand millions people, suffer from hypertension. Further, there is no miracle drug for treating hypertension. Relying solely on medicine cannot completely cure hypertension. Rather, the daily routine of an individual and the monitoring of one's blood pressure can more effectively lower the threats of hypertension. A blood pressure monitoring apparatus is one important approach to monitor one's health condition. Blood pressure is a good indicator of the condition of the cardiovascular system. A long term monitoring of the blood pressure can serve as a basis of one's health condition.

Traditionally, there are several types of blood pressure measuring apparatus, for example, the mercury type blood pressure measuring apparatus and the electronic type blood pressure measuring apparatus. The use of the mercury type blood pressure measuring apparatus normally requires a well-trained practician. However, for the sake of a higher accuracy, the mercury type blood pressure measuring apparatus is most often used in hospitals. However, the subsequent disposal and treatment of mercury is an environmental issue. Hence, many countries/regions have contemplated to ban the use of the mercury type of blood pressure measuring apparatus, and gradually, the mercury type of blood-pressure measuring apparatus is replaced by the electronic type that does not require a trained individual for operation. However, the blood pressure measuring apparatus must include an inflatable cuff. The cuff of the blood pressure measuring apparatus is wrapped around the arm (or wrist) of a subject. The cuff is then inflated to press against a desired part of the arm (or wrist) of the subject. With the pressure sensor disposed in the cuff, the blood pressure of the subject is measured.

FIG. 1A is schematic diagram of an outstretched cuff (for example, an arm cuff) of a conventional blood pressure measuring apparatus, while FIG. 1B is a schematic, side-view diagram of a conventional cuff. Referring to both FIG. 1A and FIG. 1B, the cuff 100 is provided with a strap unit 110 and a ring unit 120, wherein the ring unit 120 is sewed on the strap unit 110. When the cuff is wrapped around the arm of a subject, one end of the strap unit 110 passes through the ring and is folded back to affix to the strap unit 110. However, the direction of the ring 120 is perpendicular to outstretch direction of the strap unit 110. Thus, the strap unit 110 is in a tube shape when it is wrapped around the arm of a subject. Therefore, the cuff of a blood pressure measuring apparatus is not closely fitted around the conical-shaped upper arm of a subject. As a result, the measurement accuracy of the prior blood pressure measuring apparatus provided with the above-mentioned cuff 100 is adversely affected.

FIG. 2 is a schematic diagram of an outstretched cuff of another conventional blood-pressure measuring apparatus. The cuff 200 is in an arc shape. Therefore, when the cuff is placed around the arm of a subject, the cuff appears cone shape and can better fit on the arm of the subject. However, the tailoring of a cuff with an arc-shaped design is not material efficient, which would result in an increase of the production cost.

### SUMMARY OF THE INVENTION

Accordingly, the present invention provides a cuff, which can be closely fit on the arm of a subject. Further, no additional production cost is required.

In accordance to the present invention, a cuff is provided, wherein the cuff comprises a strap unit and a ring unit, wherein the ring is embedded at one end of the cuff, and the direction of the ring is not perpendicular to the outstretch direction of the strap unit. Further, the strap unit is adapted to pass through the ring unit.

The cuff also comprises an air bag, an air pump unit, a first adhering region and a second adhering region. The air sac is disposed, for example, inside the strap unit. The air sac further comprises an air inlet. The air pump unit, for example, is connected to the air inlet so that air can be delivered from the air inlet into the air bag. The first adhering region is disposed, for example, on the surface of the strap unit, at the one end far away from the ring, while the second adhering region is disposed on the surface of the strap unit adjacent to the first adhering region.

In accordance to the present invention, a blood pressure measuring apparatus is provided, wherein the blood pressure measuring apparatus mainly includes a blood pressure display unit, a cuff, and a pressure sensing unit. The cuff is mainly formed with a strap unit and a ring unit. The ring unit is embedded at one end of the strap unit, and the direction of the ring is not perpendicular to the outstretch direction of the strap unit. Moreover, the strap unit is adapted to pass through the ring. The pressure sensing unit is disposed on the strap, wherein the pressure sensing unit senses the pressure inside the cuff.

The cuff also includes, for example, an air bag, an air pump unit, a first adhering region, and a second adhering region. The air bag is disposed, for example, inside the strap unit. The air bag includes an air inlet. The air pump unit, for example, is connected to the air inlet for delivering air into the air bag. The first adhering region is disposed, for example, on the surface of the strap unit, at the one end further away from the ring, while the second adhering region is disposed on the surface of the strap unit adjacent to the first adhering region. The blood pressure display unit is, for example, a mercury type blood pressure display unit or an electronic type blood pressure display unit.

It is to be understood that both the foregoing general description and the following detailed description are exemplary, and are intended to provide further explanation of the invention as claimed.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention.

FIG. 1A is schematic diagram of an outstretched cuff (for example, an arm cuff) of a conventional blood pressure measuring apparatus and FIG. 1B is a schematic, side-view diagram of a prior cuff in used.

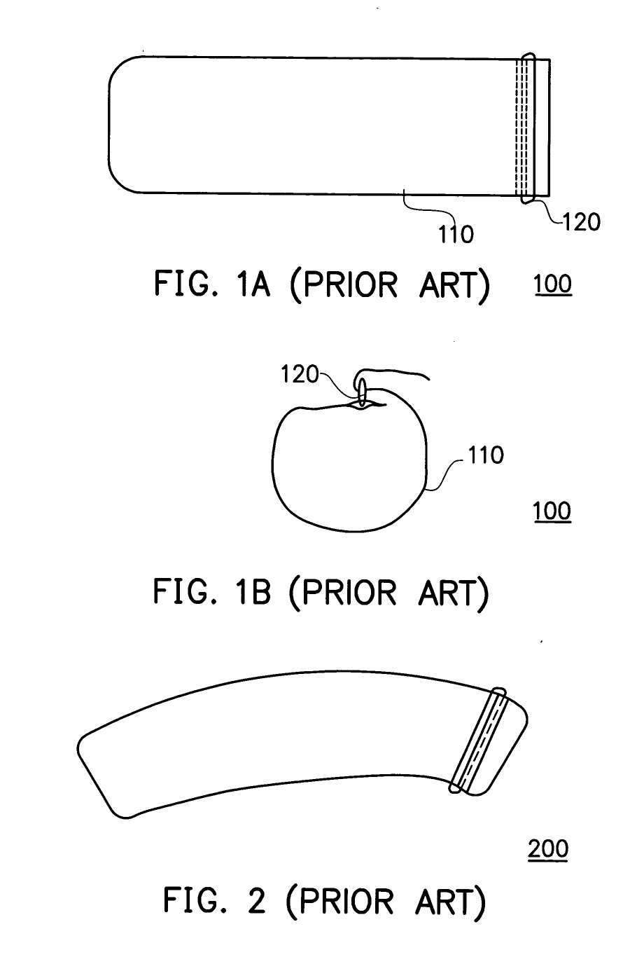

FIG. 2 is a schematic diagram of an outstretched cuff of another conventional blood-pressure measuring apparatus.

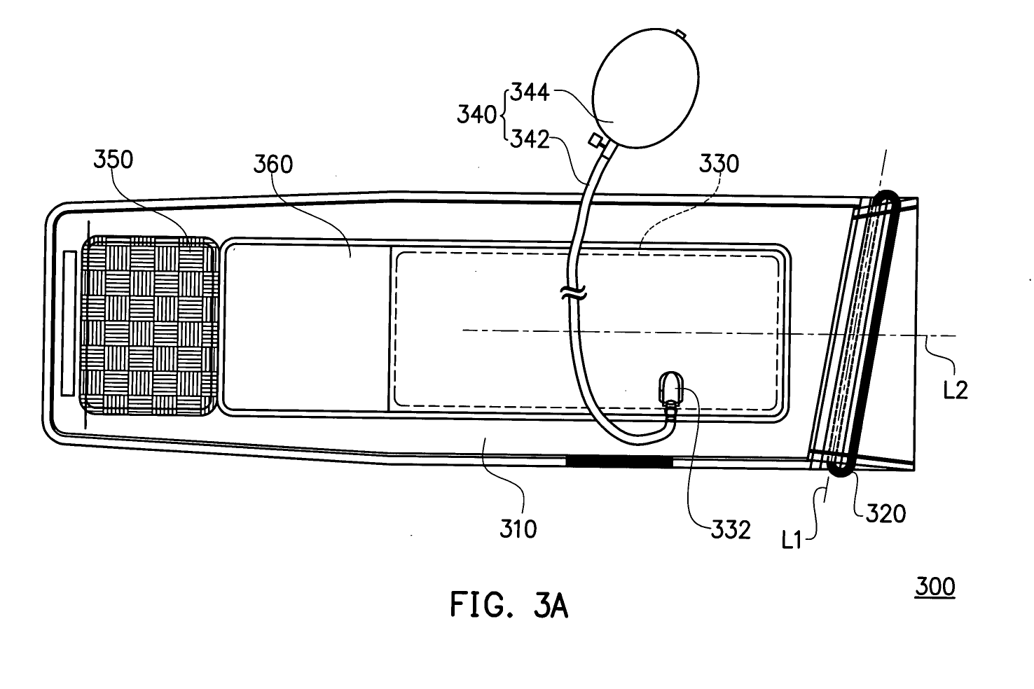

FIGS. 3A and 3B are schematic diagrams of an outstretched cuff of a blood-pressure measuring apparatus according to the present invention.

FIGS. 4A and 4B are schematic diagrams of a blood-pressure measuring apparatus provided with the above-mentioned cuff according to the present invention.

### DESCRIPTION OF THE PREFERRED EMBODIMENTS

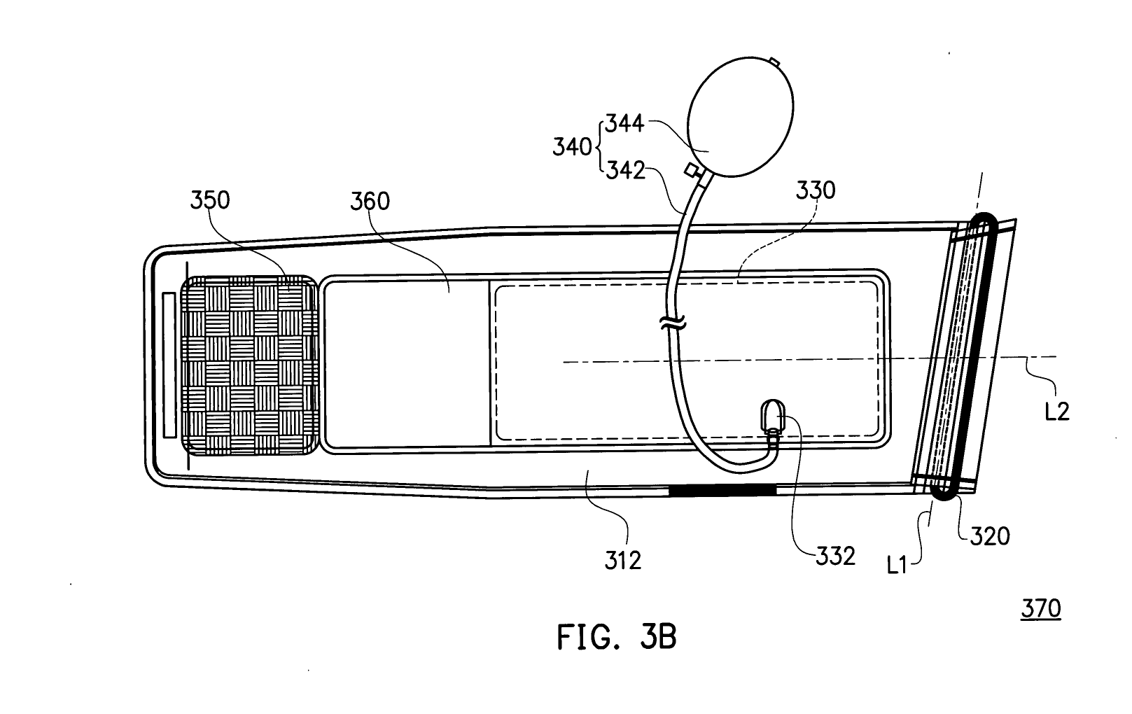

FIGS. 3A and 3B are schematic diagrams of an outstretched cuff of a blood-pressure measuring apparatus according to the present invention. Referring to FIG. 3A, a cuff 300 is mainly provided with a strap unit 310 and a ring unit 320, wherein the strap unit 310 is made of a stretchable nylon material to ensure the durability. The ring unit 320 is along a direction L1 of a straight line and embedded in the strap unit 310. The straight line is extended in a direction L1 that is not perpendicular to the outstretched direction L2 of the strap unit 310. Further, the strap unit 310 is adapted to pass through the ring unit 320. The ring unit 320 is made of, for example, a steel material, and the ring unit 320 is embedded in the strap unit 310 by means of, for example, sewing.

Further, the cuff 300 includes, for example, an air bag 330, an air pump unit 340, a first adhering region 350 and a second adhering region 360, wherein the air bag 330 is, for example, disposed inside the strap unit 310 or directly sewed on the strap unit 310. The air bag 330 includes an air inlet 332. The air pump unit 340, for example, is connected to the air pump opening 332. The air pump unit 340 is provided with, for example, air tube 342 and a pressure bulb 344. After the user presses the pressure bulb 344, air is delivered to the air bag 330 through the air tube connected to the air inlet 332. The pressure bulb 344 may be replaced by an electrical air pump (not shown).

Further, the first adhering region 350, for example, is disposed on the surface of the strap unit 310 further away from the ring unit 320 than the second adhering region 360, while the second adhering region 360 is disposed on the surface of the strap unit 310 adjacent to the first adhering region 350.

When the cuff 300 is in use, the cuff is wrapped around the arm of the subject, wherein the first adhering region 350 and the second adhering region 360 disposed on the strap unit 310 are facing the outside. One end of the strap 310 passes through the ring and is folded back for the first adhering region 350 to affix to the second adhering region 360. Further, the cuff 300 is closely wrapped around the arm of the subject. Moreover, since the ring 320 is fastened to the strap unit 310 along a direction of a straight line L1 which is not perpendicular to the outstretch direction L2 of the strap unit 310, the cuff 300 can be closely and tightly wrapped around the arm of the subject to increase the blood-pressure measuring accuracy via using the cuff 300.

Referring to FIG. 3B, the cuff 370 in FIG. 3B is provided with the ring 320 firmly fastened to the strap unit 312, along a direction L1 of a straight line, wherein the direction L1 of the straight line is not perpendicular to the outstretch direction of the strap unit 312.

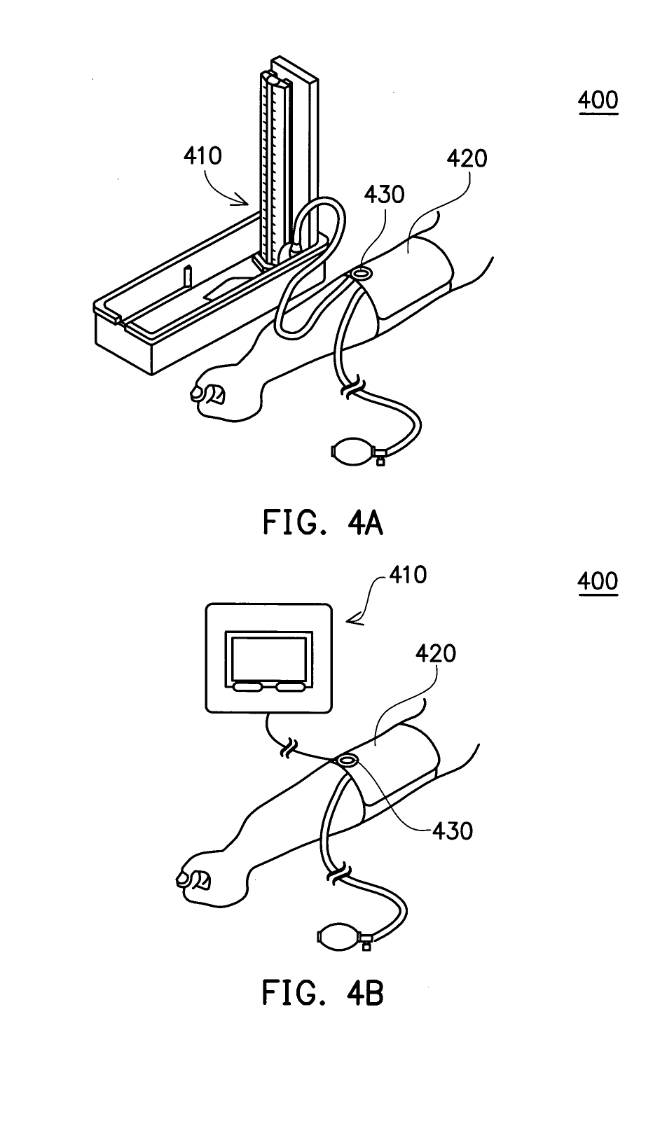

FIGS. 4A and 4B are schematic diagrams of a blood-pressure measuring apparatus provided with the above-mentioned cuff according to the present invention. Referring to FIGS. 4A and 4B, the blood-pressure measuring apparatus 400 includes a blood pressure display unit 410, a cuff 420, a pressure sensing unit 430, wherein the cuff 420 may be the cuff 300 or the cuff 370 as shown in FIGS. 3A or 3B. The pressure sensing unit 430 is disposed on the strap unit 420 to sense the pressure in the cuff 400, and outputs the pressure readings to the calculating unit (not shown) inside the blood pressure measuring apparatus. Then, the blood pressure determined by the calculating unit is shown on the display unit 410.

Further, the blood pressure display unit 410 is, for example, a mercury column, as shown in FIG. 4B or an LCD-type display unit, as shown in FIG. 4B.

Based on the foregoing description, the cuff 420 of the blood pressure measuring apparatus 400 is in a cone shape when wrapped around an arm of a subject. Therefore, the cuff 420 can be closely and tightly fitted around the arm to increase the measuring accuracy of the blood pressure measuring apparatus.

An important point to note is that the cuff of the present invention is not limited to use for an arm of a subject. The cuff can be redesigned in dimensions to be used for a subject's wrist, finger or other extremities where blood pressure measuring is easily accessible.

Accordingly, the cuff of a blood pressure measuring apparatus of the present invention is ergonomically designed, which can be closely fitted on the body extremity of a subject to increase the blood pressure measuring accuracy. The outstretch of the cuff is the typical long stripe, which is material efficient and cost effective. Further, since only the position of the ring being affixed to the strap is altered, only a slight adjustment to the production cost is required

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims and their equivalents.

1. A cuff, comprising:
a strap unit; and
a ring attached to one end of the strap unit, wherein the direction of the ring is not perpendicular to an outstretch direction of the strap unit, and the strap unit is adapted to pass through the ring.

2. The cuff of claim 1, further comprising an air sac disposed inside the strap unit, the air sac comprising an air inlet.

3. The cuff of claim 2, further comprising an air pump unit, connected to the air-pump opening, wherein air is delivered through the air inlet into the air sac.

4. The cuff of claim 1, further comprising a first adhering region and a second adhering region, wherein the first adhering region is disposed on a surface of the strap at one end further away from the ring than the second adhering region, and the second adhering region is disposed on the surface of the strap adjacent to the first adhering region.

5. A blood pressure measuring apparatus comprising:
a blood pressure display unit;
a cuff, comprising
a strap unit; and
a ring fastened on one end of the strap, wherein the direction of the ring is not perpendicular to an outstretch direction of the strap unit, and the strap unit is adapted to pass through the ring;
a pressure sensing unit, disposed on the strap unit, for sensing the pressure inside the cuff.

6. The apparatus of claim 5, further comprising an air sac, disposed inside the strap unit, and the air sac comprising an air inlet.

7. The apparatus of claim 6, wherein the apparatus further comprises an air pump unit, connected to the air pump opening, wherein air is delivered through the air inlet into the air sac.

8. The apparatus of claim 5, further comprising a first adhering region and a second adhering region, wherein the first adhering region is disposed on a surface of the strap unit at one end further away from the ring than the second adhering region, and the second adhering region is disposed on the surface of the strap unit adjacent to the first adhering region.

9. The apparatus of claim 5, further comprising a mercury column.

10. The apparatus of claim 5, further comprising an electronic display unit.
